(19) United States
(12) Patent Application Publication
(10) Pub. No.: US 2004/0180364 A1
(43) Pub. Date: Sep. 16, 2004

(54) Methods for analyzing global regulation of coding and non-coding RNA transcripts involving low molecular weight RNAs

(75) Inventors: Philipp Kapranov, Sunnyvale, CA (US); Dione Kampa, Mountain View, CA (US); Thomas R. Gingeras, Encinitas, CA (US); Stefan Bekiranov, Oakland, CA (US); Simon Cawley, Oakland, CA (US); Kyle B. Cole, Stanford, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/736,054

(22) Filed: Dec. 15, 2003

Related U.S. Application Data
(60) Provisional application No. 60/438,944, filed on Jan. 8, 2003.
(60) Provisional application No. 60/438,866, filed on Jan. 8, 2003.

(51) Int. Cl. C12Q 1/68

(52) U.S. Cl. 435/6

(57) ABSTRACT

In some embodiments of the invention, methods are provided to interrogate the transcriptional activity relating to RNAs of small molecular weight. The methods employ hybridization of a large number of oligonucleotide probes with nucleic acid derived from RNAs of small molecular weight.

### RELATED APPLICATIONS

This application claims priority to U.S. Provisional Application Serial No. 60/438,944, filed on Jan. 8, 2003, and U.S. Provisional Application Serial No. 60/438,866, filed on Jan. 8, 2003. This application is also related to U.S. patent application Ser. No. 10/316,518 filed on Dec. 12, 2002. All these applications are incorporated herein by reference for all purposes.

This invention was made with Government support under Contract No. N01-CO-12400 awarded by the National Cancer Institute, National Institutes of Health. The Government has certain rights in the invention.

### BACKGROUND OF THE INVENTION

This invention is related to biological assays, microarrays, and bioinformatics.

Transcription of DNA into RNA is the basic mechanisms by which cells mediate their growth, function, and metabolism. Therefore, understanding the transcriptional activities is important for uncovering the functions of the genome.

### SUMMARY OF THE INVENTION

In one aspect of the invention, a simple and comprehensive method to detect small RNA species using microarray technology is provided. The method can globally survey the small RNA population of a cell. In some embodiments of the invention, the method is based on the isolation of the sub-population of small RNAs, for example, using Qiagen RNA/DNA kit. One of skill in the art would appreciate that the method of the invention is not limited to any particular isolation method. The isolated RNAs can be labeled with any suitable methods, including direct 3′ labeling using T4 RNA ligase, with a RNA labeling agent disclosed in U.S. Provisional Patent Application Serial No. 60/395,580, which is incorporated herein by reference. The labeled RNA species can then be hybridized to a nucleic acid probe array such as a high density oligonucleotide probe array. In a preferred embodiment, the labeled RNA species are then hybridized to an Affymetrix oligonucleotide array with probes tiled regularly in the genome at the interval of fewer than 500, 100, 50, 30, 20, 10, 5, bases. In some cases, the labeled RNA sample may be hybridized with an array that tiles the genome at one base resolution.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a graphical representation of small RNAs detected on Chr22exp array.

FIG. 2 shows a Northern blot of small RNA identified by high density oligonucleotide array.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S.S.N 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays which are also described.

Nucleic acid arrays that are useful in the present invention include those that are commercially available from Affymetrix (Santa Clara, Calif.) under the brand name GeneChip®. Example arrays are shown on the Affymetrix website. The present invention also contemplates many uses for polymers attached to solid substrates. These uses include gene expression monitoring, profiling, library screening, genotyping and diagnostics. Gene expression monitoring, and profiling methods are shown in U.S. Pat. Nos. 5,800,992, 6,013,449, 6,020,135, 6,033,860, 6,040,138, 6,177,248 and 6,309,822. Genotyping and uses therefore are shown in USSN 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Patent application Nos. 09/916,135, 09/920,491, 09/910,292, and 10/013,598, which are incorporated herein by reference for all purposes.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170, which are incorporated herein by reference.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Glossary

The following terms are intended to have the following general meanings as used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

A nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases (see, e.g., U.S. Pat. No. 6,156, 501, incorporated herein by reference). The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids. See, e.g., U.S. Pat. No. 5,143,854.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about IM, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tm) for the specific sequence at a defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

Effective amount refers to an amount sufficient to induce a desired result.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful. See, e.g., Dong et al., Genome Research 11, 1418 (2001), in U.S. Pat. Nos. 6,361,947, 6,391,592, incorporated herein by reference.

A primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondrial DNA. A genomic library is a collection of DNA fragments represents the whole or a portion of a genome. Frequently, a genomic library is a collection of clones made from a set of randomly generated, sometimes overlapping DNA fragments representing the entire genome or a portion of the genome of an organism.

An allele refers to one specific form of a genetic sequence (such as a gene) within a cell or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from the father and one from the mother. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100or 1/1000members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

III. Methods For Analyzing Global Regulation Of Coding And Non-Coding RNA Transcripts Involving Low Molecular Weight RNAs

Low-Molecular Weight (LMW) or small RNA species (less than 200 bases or 300 bases) play different key functions in the cell: they are essential for protein synthesis (transfer tRNA, small nucleolar snoRNAs, 5S and 5.8S ribosomal rRNAs), maintenance of chromosomal structure (RNA component of telomerase), processing and maturation of messenger mRNA (smRNAs), protein localization (7.5S RNA) and many others. Recently however, they have emerged as a novel and essentially unexplored class of regulatory molecules in a cell. These molecules have been implicated in silencing genes either by specific targeted degradation of corresponding mRNAs or decreasing the rate of protein synthesis from specific mRNAs. The gene silencing mechanisms are highly evolutionary conserved from molds to humans suggesting their basic importance in a cell. The high sequence specificity mediated by small RNAs made this type of gene silencing the most promising currently-available tool to modulate gene expression in a variety of organisms, including humans. For additional discussion of small RNAs, see, e.g., Gottesman, S. (2002) Stealth regulation: Biological circuits with small RNA switches. Genes and Dev. 16: 2829-2842; Huttenhofer, A., Brosius, J., and Bachellerie, J. P. (2002) RNomics: identification and function of small, non-messenger RNAs. Curr. Opin. Chem. Biol. 6:835-843; Ambros, V. (2001) MicroRNAs: tiny regulators with great potential. Cell 107: 823-826; Lagos-Quintana, M., Rauhut, R., Lendeckel, W. and Tuschl, T. (2001) Identification of novel genes coding for small expressed RNAs. Science 294: 853-858, all incorporated herein by reference.

Despite their obvious importance, identification of novel small RNA species has lagged behind. Some of the commonly used RNA isolation methods have molecular cut-offs that prevent isolation of RNAs less than 200 bases. All currently available cDNA library construction protocols are strongly biased against RNA species less then 500-600 bases. On the other hand, isolation of novel small RNAs via construction of small RNA-specific cDNA libraries is tedious, labour intensive and is hindered by the fact that by mass the known small RNAs such as tRNAs and rRNAs by far predominate the small RNA fraction in the cell.

In one aspect of the invention, a simple and comprehensive method to detect small RNA species using microarray technology is provided. The method can globally survey the small RNA population of a cell. In some embodiments of the invention, the method is based on the isolation of the sub-population of small RNAs, for example, using Qiagen RNA/DNA kit or Ambion's mirVana™ mRNA Isolation Kit. One of skill in the art would appreciate that the method of the invention is not limited to any particular isolation method.

The isolated RNAs can be labeled with any suitable methods, including direct 3′ labeling using T4 RNA ligase, with a RNA labeling agent disclosed in U.S. Provisional Patent Application Serial No. 60/395,580, which is incorporated herein by reference. The preferred structure of a labeling agent is:

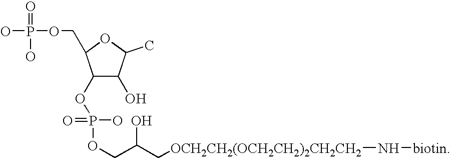

The labeled RNA species can then be hybridized to a nucleic acid probe array such as a high density oligonucleotide probe array. In a preferred embodiment, the labeled RNA species are then hybridized to an Affymetrix oligonucleotide array with probes tiled regularly in the genome at the interval of fewer than 500, 100, 50, 30, 20, 10, 5, bases. In some cases, the labeled RNA sample may be hybridized with an array that tiles the genome at one base resolution.

Genome tiling arrays and their uses in detecting transcriptional activitity are described in, for example, U.S. patent application Ser. No. 10/316,518, incorporated herein by reference.

In a large scale small RNA profiling experiment employing an exemplary embodiment (see the Example section), it was found that small RNAs are universally found along the genome. A majority of spliced and unspliced RNA transcripts encoded in the genome have at least one corresponding small anti-sense RNA transcript. Small RNAs are found in both nuclear and cytosolic compartments. Small RNAs for the same region of the same gene demonstrate differential expression patterns. They are usually found overlapping (sense or anti-sense) a larger spliced or un-spliced transcript. At any one location where a small RNA is found, there is usually no corresponding small RNA transcript on the other strand. Locations of small RNA transcripts are many times found at the exon-intron junctions, or splice sites, and thus, may be such small RNA molecule can be an important participant in the processing of RNAs. Other possible roles for these RNAs include stabilizing (i.e. effect turnover) or destabilizing larger coding and non-coding transcripts, influencing (positive and negative) translation processes of larger coding transcripts, assisting in subcellular localization, influencing (positive and negative) the transport of specific larger transcripts to specified subcellular regions, assisting or inhibiting transcription of larger coding and non-coding transcripts, modifying chromatin, modifying DNA in the regions encompassing larger coding and non-coding transcripts and assisting in the editing of larger coding transcripts.

In another aspect of the invention, the small RNA activity profiling using the methods of the invention may be employed for clinical diagnostics. In such applications, a small RNA profile obtained from a patient sample may be compared with one or more reference profiles (diseased or normal) to detect the similarity of the transcriptional activity pattern with the reference profiles. The reference profiles may be obtained by interrogating diseased and normal tissues for transcriptional activity using the methods of the invention.

Small RNA activity profiling may be also used for in vitro toxicity testing. In such applications, a chemical compound is used to treat a cell culture. The small RNA activity of the cells may be interrogated. The profile of small RNA activity may be compared with reference profiles to detect whether the compound may have toxic effects. The reference profiles may be generated by testing known toxic and nontoxic compounds for toxic and non toxic small RNA activity profiles.

Similarly, small RNA activity profiling may be used for testing drug candidates. In such applications, a drug candidate may be tested in cell cultures to determine whether it induces desirable small RNA activity.

In yet another aspect of the invention, the small RNA activity discovered using the methods of the invention may be used for designing microarrays for small RNA expression monitoring. Probes targeting small RNA may be designed and immobilized on a substrate to form a microarray that can be used to monitor the expression of the novel transcripts.

IV. Example

The following example illustrates preferred embodiments of the invention. One of skill in the art would appreciate that the example is provided to illustrate the embodiments and that the scope of the invention is not limited to the specific exemplary embodiments.

Unlabeled, low molecular weight RNA was prepared from mammalian cells using Qiagen RNA/DNA kit (Cat. No. 14162) according to the manufacturer's protocol. This fraction of total RNA from the cells ranges from ˜200 bases and below. The RNA was dephosphorylated followed by 3′ end-labeling using T4 RNA ligase and the labeling reagent described above (pCp-biotin; U.S. Provisional Application Serial No. 60/395,580, incorporated herein by reference). 15 μg of small RNA was treated with Shrimp Alkaline Phosphatase (Amersham Pharmacia) at a final concentration of 0.01 U/μl in 20 μl reactions at 37° C. for 35 minutes. The Shrimp Alkaline Phosphatase was then heat inactivated at 65° C. for 20 minutes.

The entire dephosphorylation reaction was ligated to 250 μM (pCp-biotin) with 5U/ul T4 RNA ligase (Ambion) and 12.5% PEG for 2 hours at 37° C. in 40 μl.

Each 40 μl ligation reaction was then added to a hybridization cocktail containing 50 pM control oligo B2 (Affymetrix), 50 pM control oligo 213B (Affymetrix), 1×Eukaryotic Hybridization Controls (Affymetrix), 0.1 mg/ml Herring Sperm DNA (Invitrogen), 0.5 mg/ml Acetylated BSA (Invitrogen), and 1×MES for a total volume of 300 μl. Approximately 10 μg of labeled small RNA was hybridized to Affymetrix Chr22exp sense or antisense arrays for 18 hours at 45° C. Chr22exp array interrogates ˜360 kb of DiGeorge minimal critical region of human chromosome 22 at a 1 bp resolution with 14 micron features. Standard wash and stain protocols were used as recommended in the GeneChip Expression Analysis technical manual. The arrays were scanned on the Agilent GeneArray® scanner with 2 micron pixel and 100% PMT settings.

A probe identified from the array data to be anti-sense to exon-6 in the DGSI gene was constructed and labeled with 32P using the Starfire Nucleic Acid Labeling System (Integrated DNA Technologies, Inc.) and purified using Bio-Spin 30 columns (Bio-Rad, Inc.). 30 μg of HepG2 and SK-N-AS cytoplasmic small RNA and 20 μg of CEM cytoplasmic small RNA was fractionated in a 15% acrylamide gels with 7M urea in 1×tris-borate buffer, transferred to nylon membranes (Hybond-N+, Amersham Pharmacia) in 0.5×tris-borate buffer, UV-crosslinked and baked at 80° C. for 1 hr. The filters were then hybridized to the 32P radiolabeled probe in 50% formamide, 5×saline sodium phosphate EDTA, 5× Denhardt's reagent, 0.5% sodium dodecyl sulphate, 80 μg/ml fragmented herring sperm DNA. The membrane was exposed to a phosphorimager screen for 4 hours and visualized using a Storm Phosphorimager (Molecular Dynamics).

A Wilcoxon Signed Rank test (M. Hollander and D. A. Wolfe) is applied to Zi where for each probe pair

Zi=log2(PMi/MMi) (1)

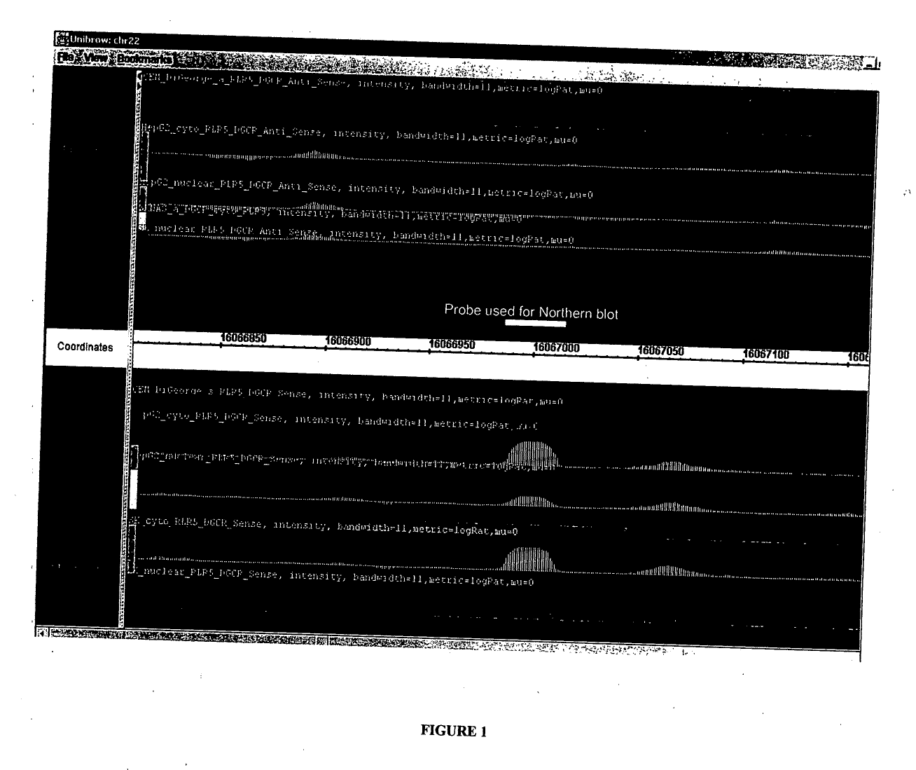

and i is the set of probe pairs within a window of +11 bases about a central base for which the p-value and Hodges-Lehmann estimator are reported. The p-value and Hodges-Lehmann estimator are calculated for every base of the DiGeorge minimal critical region tiled on the Chr22exp array using this sliding window of 23 bases. This is to apply a statistical test which tests the null hypothesis that PM (perfect match)=MM (mismatch) within a window corresponding to the size of the smallest RNA of biological interest which corresponds to ˜22 bases. If PM>MM for a significant number of probes within this window, there is a high likelihood that a transcript has been detected in the region near the central base. This will give low p-values and relatively high Hodges-Lehmann estimators as shown in the highlighted region of FIG. 1. The Hodges-Lehmann estimator is the median of all 276 pairwise averages (Zi+Zj)/2 where i<=j=1, . . . ,23.

This test can also be applied using the metric

Zi=log2(max(PM-MM,1)). (2)

The test can also be applied with different length windows.

One way to get around the “dependence” problem is by applying thresholds calculated in Bacterial Negative Control regions which correspond to false positives FP=0.01 and 0.03.

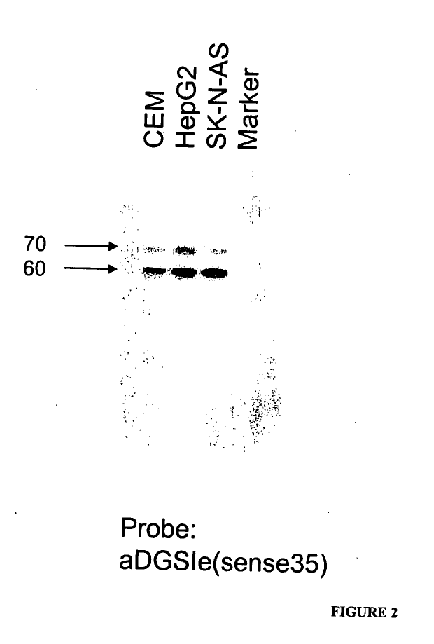

FIG. 1 is a graphical representation of small RNAs detected on Chr22exp array. The position of each bar represents the first base of a probe pair, and its height represents the corresponding Log2(PM/MM). The different tracts represent hybridization results from cytosolic or nuclear fractions of three cell lines, CCRF-CEM, HepG2 or SK-N-AS. Top and bottom 5 graphs represent results of anti-sense and sense Chr22exp arrays, respectively. In each half, the graphs are arranged as following: CCRF-CEM cytoplasmic, HepG2 cytoplasmic, HepG2 nuclear, SK-N-AS cytoplasmic, and SK-N-AS nuclear. As indicated by the arrow, a small RNA transcript from all cell lines is readily identifiable by hybridizing small RNA to high-density oligonucleotide Chr22exp-sense array. Such transcript would be anti-sense to the exon of a known gene DGS-I, shown on the picture as green or pink bar. No hybridization is seen on the anti-sense version of the same array. The probe used to detect the small RNA transcript on a Northern blot in FIG. 2 is shown as a white bar. Transcriptional fragments corresponding to the small RNA molecules are readily detected on the arrays. Furthermore, many of the hybridizing species are anti-sense to known genes, suggesting a regulatory role.

To validate this method in identifying small RNA targets on high-density oligonucleotides, a probe was constructed to identify a small RNA anti-sense to exon-6 in the DGSI gene by Northern blot. The small RNA hybridized to two transcripts of 70 and 60 bases in length, as seen in FIG. 2. The size of the transcript seen on the arrays is comparable to the size of the transcripts on the Northern.

Based upon the data using the Chr22exp array, a number of interesting observations are obtained. Small RNAs are universally found along the genome. A majority of spliced and unspliced RNA transcripts encoded in genome have at least 1 corresponding small anti-sense RNA transcript. Small RNAs are found in both nuclear and cytosolic compartments. Small RNAs for the same region of the same gene demonstrate differential expression patterns. They are usually found overlapping (sense or anti-sense) a larger spliced or un-spliced transcript. At any one location where a small RNA is found, there is usually no corresponding small RNA transcript on the other strand. Locations of small RNA transcripts are many times found at the exon-intron junctions, or splice sites, and thus, may be such small RNA molecule can be an important participant in the processing of RNAs. Other possible roles for these RNAs include stabilizing (i.e. effect turnover) or destabilizing larger coding and non-coding transcripts, influencing (positive and negative) translation processes of larger coding transcripts, assisting in subcellular localization, influencing (positive and negative) the transport of specific larger transcripts to specified subcellular regions, assisting or inhibiting transcription of larger coding and non-coding transcripts, modifying chromatin, modifying DNA in the regions encompassing larger coding and non-coding transcripts and assisting in the editing of larger coding transcripts.

V. Conclusion

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method of determining small RNA transcriptional activity comprising:
obtaining a small RNA sample;
hybridizing the small RNA or nucleic acids derived from the RNA with an oligonucleotide probe array, wherein the oligonucleotide probe array contains at least 10,000 perfect match (PM) probes, each of the perfect match probes targeting a different transcript sequence from a region of a genome; and
determining that a genomic sequence is transcribed if the probe against the genomic sequence is hybridized with a target.

2. The method of claim 1 wherein the region of the genome is at least 20 MB

3. The method of claim 2 wherein the region of the genome is at least 50 MB.

4. The method of claim 3 wherein the region of the genome is 25% of the DNA sequences in a chromosome.

5. The method of claim 4 wherein the region of the genome is 50% of the DNA sequences in a chromosome.

6. The method of claim 5 wherein the region of the genome is the DNA from a chromosome.

7. The method of claim 6 wherein the region of the genome is the DNA sequence from the entire genome.

8. The method of claim 2 wherein the probes target the transcript sequences from the genome at a resolution of at least 100 bps.

9. The method of claim 8 wherein the probes target the transcript sequences from the genome at a resolution of at least 30 bps.

10. The method of claim 9 wherein the probes target the transcript sequences from the genome at a resolution of at least 10 bps.

11. The method of claim 10 wherein the probes target the transcript sequences from the genome at the resolution of 1 bp.

12. The method of claim 2 wherein the small RNA sample is obtained from the nuclei.

13. The method of claim 2 wherein the small RNA sample is obtained from the cytoplasm.

14. The method of claim 13 wherein the oligonucleotide probe array contains at least 100,000 oligonucleotide probes, each targeting a transcript sequence from a different region of a genome.

15. The method of claim 14 wherein the oligonucleotide probe array contains at least 500,000 oligonucleotide probes, each targeting a transcript sequence from a different region of a genome.

16. The method of claim 15 wherein the oligonucleotide probe array contains at least 800,000 oligonucleotide probes, each targeting a transcript sequence from a different region of a genome.

17. The method of claim 2 wherein oligonucleotide array further comprises mismatch (MM) probes, wherein each of the mismatch probes is different from a perfect match probe in one base.

18. The method of claim 17 wherein the mismatch probes is different from the perfect match probe in a middle position.

19. The method of claim 2 wherein the perfect match probes are targeting transcripts from non-repetitive sequence of the genome.

20. A method for comparing the small RNA transcriptional activity of two biological samples comprising:
obtaining a first small RNA sample;
obtaining a second small RNA sample;
hybridizing the first and second small RNA samples or nucleic acids derived from the first and second small RNA with an oligonucleotide probe array wherein the oligonucleotide probe array contains at least 10,000 perfect match (PM) probes, each of the perfect match probes targeting a different transcript sequence from a region of a genome;
determining, for each of the first and second sample, that a genomic sequence is transcribed if the probe against the genomic sequence is hybridized with a target; and
comparing the transcribed sequences between the first and second sample.
